(19) United States
(12) Patent Application Publication
(10) Pub. No.: US 2026/0048164 A1
(43) Pub. Date: Feb. 19, 2026

(54) Always-in Chemical Indicator Assurance System

(71) Applicant: EDUARDO L. PEREZ, PORT SAINT LUCIE, FL (US)

(72) Inventor: EDUARDO L. PEREZ, PORT SAINT LUCIE, FL (US)

(21) Appl. No.: 18/803,230

(22) Filed: Aug. 13, 2024

(51) Int. Cl. A61L 2/28

(52) U.S. Cl. CPC A61L 2/28; A61L 2202/14; A61L 2202/24

(57) ABSTRACT

The Always-in Chemical Indicator Assurance System is a metal device that can be attached to any surgical sterilization tray to ensure chemical indicator compliance, validate the sterilization process, and provide a clear reading of the internal chemical indicator. The device is designed to force sterile processing technicians to include the required chemical indicators inside the tray before closing the surgical tray lid in preparation for the sterilization process. The device will not permit the completion of a surgical tray without adding a chemical indicator because the device will obstruct the sterilization tray lid attachment. The device has a universal indicator attachment opening that permits the sterile processor to insert the chemical indicator, turning the indicator into a locking mechanism that keeps the device down without obstructing the upper border of the tray, allowing the sterile processing tech to attach the sterilization tray lid meeting the indicator compliance requirements.

### CROSS REFERENCE

Provisional Application No. 63/568,424

### BACKGROUND OF THE INVENTION

### Brief Summary of the Invention

The always-in chemical indicator assurance system is a small rectangular metal device contraction that can be attached to the side of any surgical tray. The device has a mechanism that is activated when the chemical indicator is not inserted in the chemical indicator channel; the device will eject a metal sheet at the top exterior side of the device, creating a physical interference with the surgical tray lid attachment and preventing the conclusion of the prep and pack sterilization process without adding the proper chemical indicator inside the tray.

### Detailed Description of the Invention

The always-in chemical indicator assurance device is a metal device with a tray attachment clip that can be attached to any surgical tray container or inner basket and adjusted to different sizes of sterilization containers. The device comprises two metal body parts, a metal sheet, one indicator insertion channel, an internal ejector spring, and one pressure-locking mechanism.

The device is designed to assure compliance with the chemical indicator requirement needed to verify the sterility of surgical trays used in surgical procedures. The device mechanism is designed to eject a metal sheet at the top exterior part of the device that will interfere with the sterile container lid closing if the chemical indicator is not inserted in the device indicator insertion channel, providing a hard stop to the sterilization technician by not permitting the closure and conclusion of the sterile packing and preparation process prior to sterilization.

### SPECIFICATION

### Description

### Drawings Specification

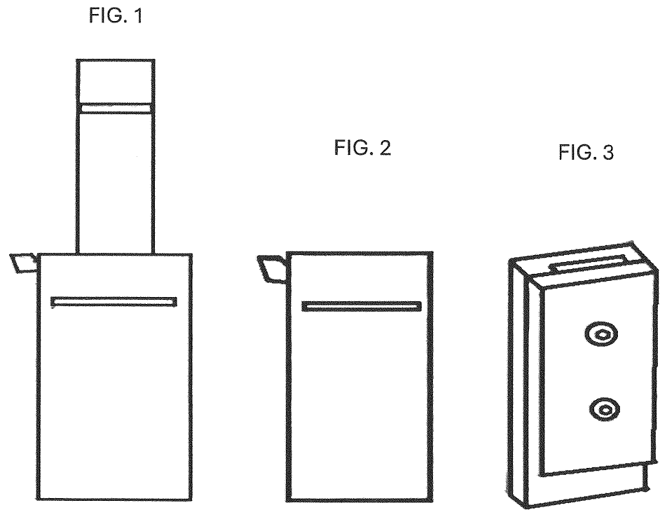

### FIG. 1

The image shows the always-in chemical indicator assurance system device's front side in the open locked position without a chemical indicator. In the absence of the chemical indicator, the metal sheet ejects up and gets locked, with the side locking mechanism impeding the metal sheet from sliding down, providing a physical obstruction of the tray lid. The sheet will be in the interior of the device and will slide up and down when the side locking button is pressed to release the open locked position.

### FIG. 2

This image shows the always-in chemical indicator assurance system device in the closed, locked position. To activate the close lock position, the user must push the side button to slide down the metal sheet and insert a chemical indicator in the chemical indicator channel as the locking (agent) securing the metal sheet is locked in place and hidden inside the device. This will allow the user to close the surgical tray lid without any obstruction for processing completion and will assure the compliance with the chemical indicator is inside the surgical tray required to verify the effectiveness of the sterilization process.

### FIG. 3

This image is a side view of the always-in chemical indicator assurance system device to demonstrate the device attachment clip that can be attached to any exterior or interior surgical tray container. The clip will have adjusting screws to fixate the device to any size or type of sterilization container variety. The adjustable clip will allow the device to be attached to inner baskets on rigid close containers. It will also allow the device to be attached to the side of any non-close container, providing chemical sterilization assurance to any surgical tray rigid close container or non-close container.

1. The Always-in Chemical Indicator Assurance System device comprises:
An attachment clip can be attached to any surgical tray or sterilization container.
A metal sheet ejector mechanism with a pressure locker to provide the functionality of interfering with the addition of the sterilization container lid if the chemical indicator is not inserted in the device to assure the sterilization processor complies with the chemical indicator use.
A chemical indicator channel that permits the insertion of a chemical indicator to act as a locking mechanism for the device, permitting the sterile processor to attach and close the container lid without obstruction.

2. The Always-in Chemical Indicator Assurance System device as in claim 1, is the only device with the functionality to ensure that sterile processors cannot close the surgical tray without adding the required chemical indicator inside.

3. The Always-in Chemical Indicator Assurance System device as in claim 1, is the only device designed to provide a hard stop to the sterilization tech by interfering with the tray lid attachment if the chemical indicator is not added inside the sterile tray.

4. The Always in Chemical Indicator Assurance System as in claim 1, will ensure the compliant use of sterilization chemical indicators inside surgical trays to verify the sterilization process.

5. The Always-in Chemical Indicator Assurance System device as in claim 1, is designed to work with any type of chemical indicator, including Steam, ETO, and low-temperature GAS Plasma.

6. The Always-in Chemical Indicator Assurance System device as in claim 1, is designed to be adjusted to any size or type of sterilization container. It will attach to Closed rigid containers and or to non-close surgical tray containers sterile wrap-required containers.

7. The Always-in Chemical Indicator Assurance System device as in claim 1, provides a clear, unobstructed view for surgical techs and surgical services staff to read the chemical indicators' results. Because of the location of the device in reference to the view of the user at the time of opening the sterile tray.
